(19) United States
(12) Patent Application Publication
(10) Pub. No.: US 2026/0256147 A1
(43) Pub. Date: Sep. 3, 2026

(54) METHOD FOR IMPROVING PLANT DROUGHT TOLERANCE, SERICIN PROTEIN WORKING SOLUTION AND APPLICATION THEREOF

(71) Applicant: Zhejiang A&F University, Hangzhou (CN)

(72) Inventors: Li TIAN, Hangzhou (CN); Yali YAN, Hangzhou (CN); Laining ZHANG, Hangzhou (CN)

(21) Appl. No.: 19/428,131

(22) Filed: Dec. 20, 2025

(30) Foreign Application Priority Data
Jan. 7, 2025 (CN) 2025100184662

(51) Int. Cl. A01N 63/50; A01N 25/02; A01P 21/00; C07K 14/435

(52) U.S. Cl. CPC A01N 63/50; A01N 25/02; A01P 21/00; C07K 14/43586

(57) ABSTRACT

A method for improving plant drought tolerance, a sericin protein working solution and an application thereof are provided, which relate to the field of plant cultivation technologies. The method includes: preparing a sericin protein working solution with a concentration of 0.1 g/L to 0.5 g/L, and spraying the sericin protein working solution onto foliage of tomato plants. The method for improving plant drought tolerance significantly improve a survival rate, a biomass, and a water content of the tomato plants under drought stress conditions.

### CROSS-REFERENCE TO RELATED APPLICATION

This application claims priority to Chinese Patent Application No. 202510018466.2, filed on Jan. 7, 2025, which is herein incorporated by reference in its entirety.

### TECHNICAL FIELD

The disclosure relates to the field of plant cultivation technologies, and more particularly to a method for improving plant drought tolerance, a sericin protein working solution and an application thereof.

### BACKGROUND

Persistent high-temperature weather and reduced rainfall cause varying degrees of drought stress on plants, which severely affect crop growth and agricultural production. The causes of drought stress are manifold. For example, high temperature, intense light, and low atmospheric humidity may lead to strong plant transpiration, thereby resulting in substantial water loss; a lack of available water in the soil makes water absorption difficult for plants, thereby causing water deficit; factors such as excessively low soil temperature, hypoxia, high ion concentration, or accumulation of toxic substances can impede the physiological activities of plant roots, thereby preventing normal water uptake and resulting in physiological drought.

Drought stress is primarily manifested by a decrease in plant water content, thereby leading to water deficit, which causes damage to plant growth. The damage to plant growth includes hindering plant cell division, inhibiting plant cell elongation, reducing photosynthesis, impeding protein synthesis, causing metabolic disorders, slowing plant growth, obstructing development, causing senescence and abscission of plant organs, and even causing overall wilting and death of the plant. How to enhance plant tolerance to drought stress is an urgent problem to be solved.

### SUMMARY

In order to improve plant tolerance to drought stress, the disclosure provides a method for improving plant drought tolerance and an application method of a sericin protein working solution. The method for improving drought tolerance in tomato plants provided by the disclosure significantly increases the survival rate, biomass, and water content of tomato plants under drought stress conditions.

The disclosure provides a method for improving plant drought tolerance, including the following steps:

Preparation of sericin protein working solution: Silkworm cocoons are fragmented and the fragmented silkworm cocoons are boiled in distilled water for 10 minutes (min) to 15 min to obtain a sericin protein stock solution; the sericin protein stock solution is diluted with distilled water to a concentration of 0.1 gram per liter (g/L) to 0.5 g/L, to obtain the sericin protein working solution.

The sericin protein solution is sprayed onto adaxial surfaces of leaves of the tomato plant when the tomato plant grows to a five-true-leaf and one-heart stage, with a spray volume of 10 mL/plant to 15 mL/plant.

Silk protein fibers are primarily composed of an inner fibroin core coated with an outer sericin layer, with the outer sericin component accounting for approximately 20%. As a natural protein material, sericin protein possesses good moisturizing, antioxidant, and anti-aging properties, and has been used in numerous fields such as cosmetics, food, and biomedical materials. There are few reports on the application of sericin protein in agriculture. The disclosure is the first to use sericin protein as a foliar spray agent on plant leaves to enhance plant drought tolerance, improve the survival rate of plants under drought stress, increase root biomass and water content, aid in seedling preservation during drought, and reduce the risks posed by drought to crop growth and yield.

In an embodiment, the spraying timing is set when the tomato plant grows to the five-true-leaf and one-heart stage, and the spraying operation is performed at a time point when a cultivation environment changes from darkness to light.

The cultivation environment involves repeated cycles of 16 hours (h) of light and 8 h of darkness.

In an embodiment, during the process of fragmenting silkworm cocoons and boiling the fragmented cocoons in distilled water, the mass-to-volume ratio of the silkworm cocoons to the distilled water is 5 to 10 grams (g): 200 milliliters (mL).

In an embodiment, the concentration of the sericin protein working solution is 0.1 g/L.

The disclosure further provides a sericin protein working solution, which is prepared by the aforementioned steps.

The disclosure further provides an application method of the sericin protein working solution in tomato plant cultivation, and the sericin protein working solution is used to improve drought tolerance of tomato plants.

In an embodiment, the sericin protein working solution is configured to improve a survival rate of the tomato plants under drought stress.

In an embodiment, the sericin protein working solution is configured to improve a root biomass and a root water content of the tomato plants under drought stress.

In an embodiment, the sericin protein working solution is configured to improve a shoot biomass and a shoot water content of the tomato plants under drought stress.

In an embodiment, the tomato plant is wild-type tomato Ailsa craig.

Compared with the related art, the beneficial effects of the disclosure are as follows:

The disclosure is the first to use sericin protein as a foliar spray agent on tomato plant leaves, which increases the survival rate, root biomass, root water content, shoot biomass, and shoot water content of tomato plants under drought stress. The method of the disclosure improves plant drought tolerance, aids in seedling preservation during drought, reduces the risks posed by drought to crop growth and yield, and can be utilized to develop applications aimed at saving irrigation water by enhancing plant drought tolerance.

### BRIEF DESCRIPTION OF DRAWINGS

To more clearly illustrate embodiments of the disclosure or technical solutions in the related art, the drawings required for describing the embodiments or the related art will be briefly introduced below. Obviously, the drawings in the following description are merely some of the embodiments of the disclosure, and those skilled in the art can obtain other drawings based on these drawings without exerting creative effort.

FIG. 1A illustrates a schematic diagram of phenotypic effects of different treatments on tomato plants; NS group represents no spray of any substance, HS group represents foliar spray of distilled water, and DS group represents foliar spray of a sericin protein working solution.

FIG. 1B illustrates a schematic statistical diagram of effects of treatments in the NS group, the HS group, and the DS group on a survival rate of the tomato plants.

FIG. 2A illustrates a schematic diagram of an effect of the sericin protein working solution on a fresh weight of root of the tomato plants under drought stress.

FIG. 2B illustrates a schematic diagram of an effect of the sericin protein working solution on a dry weight of root of the tomato plants under drought stress.

FIG. 2C illustrates a schematic diagram of an effect of the sericin protein working solution on a water content of root of the tomato plants under drought stress.

FIG. 2D illustrates a schematic diagram of an effect of the sericin protein working solution on relative decrease in plant biomass (RDPB) of the fresh weight of root of the tomato plants under drought stress.

FIG. 2E illustrates a schematic diagram of an effect of the sericin protein working solution on relative decrease in plant biomass of the dry weight of root of the tomato plants under drought stress.

FIG. 2F illustrates a schematic diagram of an effect of the sericin protein working solution on a fresh weight of shoot of the tomato plants under drought stress.

FIG. 2G illustrates a schematic diagram of an effect of the sericin protein working solution on a dry weight of shoot of the tomato plants under drought stress.

FIG. 2H illustrates a schematic diagram of an effect of the sericin protein working solution on a water content of shoot of the tomato plants under drought stress.

FIG. 2I illustrates a schematic diagram of an effect of the sericin protein working solution on relative decrease in plant biomass of the fresh weight of shoot of the tomato plants under drought stress.

FIG. 2J illustrates a schematic diagram of an effect of the sericin protein working solution on relative decrease in plant biomass of the dry weight of shoot of the tomato plants under drought stress.

### DETAILED DESCRIPTION OF EMBODIMENTS

The specific embodiments of the disclosure will be described in detail below, but it should be understood that a scope of protection of the disclosure is not limited by the specific embodiments. Based on the embodiments of the disclosure, all other embodiments obtained by those skilled in the art without creative labor are within the scope of protection of the disclosure. The experimental methods described in each embodiment of the disclosure are conventional methods unless otherwise specified. The materials and reagents used in the following embodiments can be obtained from commercial sources unless otherwise specified.

### Embodiment 1

### I. Experimental Preparation

### 1. Preparation of Tomato Plants

In the embodiment, tomato plants are used as the test plant material. A variety of the tomato plants is wild-type tomato Ailsa craig. Tomato seeds are disinfected sequentially with 70% (volume to volume, abbreviated as v/v) ethanol for 30 seconds(s) and 10% (v/v) sodium hypochlorite for 10 min, rinsed five times with sterile water, dried on sterile filter paper, and then placed in a 28 Celsius degrees (°C) incubator for dark cultivation until germination. The germinated tomato seeds are transplanted into pots and placed in a light incubator for cultivation. The cultivation is divided into two stages. Stage 1 is set with a light intensity of 22000 lux (lx), a temperature of 25° C., and a duration of 16 h. Stage 2 is set under complete darkness, with a temperature of 18° C., and a duration of 8 h. Subsequent experiments are carried out when the tomato plants grow to the five-true-leaves and one-heart stage (five expanded leaves and one apical bud). The cultivation conditions for the subsequent experiments cyclically repeated between the stage 1 and the stage 2.

### 2. Preparation of Sericin Protein Working Solution

An appropriate amount of silkworm cocoons is taken. After removing the outermost and innermost silk fiber layers, the clean middle portion of the silkworm cocoons is used to prepare a sericin protein stock solution. 5 g of clean silkworm cocoons are fragmented into pieces, placed in 200 mL of distilled water, and boiled for 10 min. The remaining undissolved fibroin fiber is removed by filtration. The resulting solution is the sericin protein stock solution. The concentration of the sericin protein stock solution is determined by the bicinchoninic acid (BCA) method and is diluted with distilled water to a concentration of 0.1 g/L to obtain the sericin protein working solution for subsequent experiments.

### II. Experimental Design

The tomato plants at the five-true-leaves and one-heart stage with uniform growth and size are selected for experimental treatment. Three groups of experimental plants are set up, which are labeled as a NS group, a HS group, and a DS group, with 15 plants in each group. The NS group is the no-spray control group, meaning no substance is sprayed on the leaf surfaces. The HS group is the distilled water group, meaning distilled water is sprayed on the adaxial surfaces of leaves. The DS group is the sericin protein group, meaning the sericin protein working solution is sprayed on the adaxial surfaces of leaves.

Spray treatment is conducted according to the above groups. The spraying procedure is as follows. At a time point when the light incubator switched from darkness to light, a uniform spray is applied to the adaxial surfaces of leaves at a volume of 10 mL/plant, repeated every 2 days.

Drought simulation treatment is performed. Drought stress is simulated by applying a 20% (weight to volume, abbreviated as w/v) polyethylene glycol (PEG 6000) solution. A 20% PEG 6000 solution is irrigated every 2 days for a continuous period of 12 days. Spraying and drought treatments are conducted simultaneously.

An additional group of experimental plants with uniform growth and size is set as a control under normal growth conditions without drought simulation.

After the above treatments are completed, a biomass and a water content of each tomato plant in each group are calculated. The specific methods are as follows.

The biomass and water content indicators of the tomato plants are analyzed as follows. After the above-mentioned treatment, the wilting degree of plants is observed, and the survival rate of each group is calculated. Roots and shoots of the tomato plants are separately weighed for fresh weight, then dried at 85° C. for 5 days and separately weighed for dry weight.

The obtained experimental data are calculated according to the following formula:

$$survival rate = ( {N}_{t} - {N}_{d} ) / {N}_{t} ;$$

where N_{d} represents a number of plants that did not survive; and Nₜ represents a total number of test plants.

A criterion for determining N_{d} under drought treatment is as follows. If an entire plant shows completely withered, yellowed, and wilted, with the top of the shoot being dry, and lodging occurring, the plant is recorded as a non-surviving plant (N_{d}) under drought treatment. If the entire plant remains upright, only some leaves of the shoot wilt, no lodging occurs, and the whole plant still appears green, the plant is recorded as a surviving plant under drought treatment.

Water content=(Y_{fw}-Y_{dw})/Y_{fw}; where Y_{fw} represents a fresh weight, and Y_{dw} represents a dry weight.

Relative decrease in plant biomass (%)=(Y₁-Y₂)/Y₁; where Y₁ represents a fresh weight of the control plants without drought treatment, and Y₂ represents a fresh weight of the plants subjected to drought simulation treatment.

### III. Analysis of Experimental Results

### 1. Effect of Sericin Protein Working Solution on Survival Rate of Tomato Plants Under Drought Stress

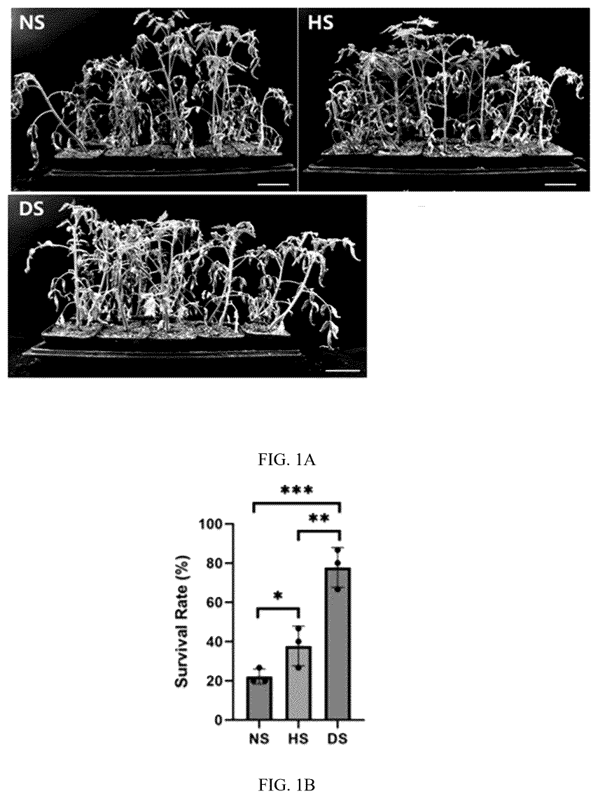

FIG. 1A shows the growth status of plants after treatment in the NS, HS, and DS groups following 12 days of drought simulation treatment. As shown in FIG. 1A, the vast majority of plants in the NS group (no spray of any substance) show overall withering, yellowing, wilting, and death, with only a very few individual plants surviving. The statistical counts are shown in FIG. 1B, with a survival rate below 20%.

Compared to the NS group (no spray of any substance), the HS group (foliar application of distilled water) shows some mitigating effect on drought damage, with a reduced number of withered, yellowed, wilted, and dead plants. The statistical counts are shown in FIG. 1B, with a survival rate of approximately 40%. The DS group (foliar application of sericin protein working solution) shows significantly enhanced drought tolerance. Although a few individual plants still show signs of withering, yellowing and wilting, the overall population grows well, remains upright, and stays green. The statistical survival rate is shown in FIG. 1B, reaching over 80%.

It can be seen that the survival rate of tomato plants sprayed with the sericin protein working solution under drought conditions is as high as over 80%. This represents a 40% increase compared to tomato plants sprayed with distilled water and a 60% increase compared to tomato plants sprayed with no substance. This indicates that spraying the sericin protein working solution can significantly improve the drought tolerance of tomatoes.

### 2. Effect of Sericin Protein Working Solution on Biomass and Water Content of Tomato Plants Under Drought Stress

### (1) Effect of Sericin Protein Working Solution on Root Biomass and Root Water Content of Tomato Plants Under Drought Stress

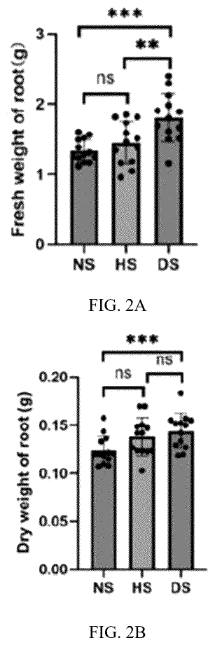

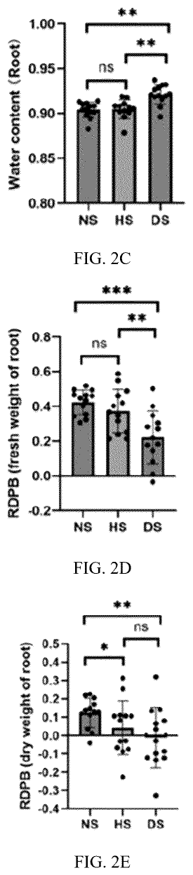

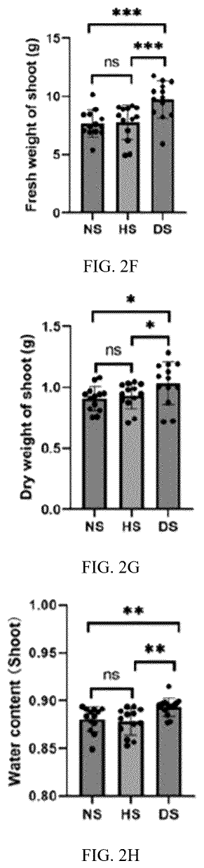

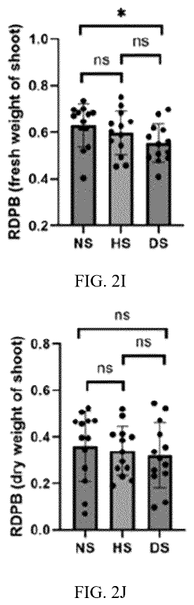

As can be seen from FIGS. 2A and 2B, the root fresh weight and dry weight of plants in the HS group (sprayed with distilled water) are slightly higher than that in the NS group (no spray of any substance), but the difference in population performance is not significant. In contrast, the root fresh weight and dry weight of plants in the DS group (sprayed with sericin protein working solution) are significantly higher than those in the NS group (no spray of any substance), and the fresh weight is significantly higher than that in the HS group (sprayed with distilled water).

As shown in FIG. 2C, there is no significant difference in root water content between the HS group (sprayed with distilled water) and the NS group (no spray of any substance). However, the root water content of plants in the DS group (sprayed with sericin protein working solution) is significantly higher than that in both the HS group and the NS group. This indicates that spraying distilled water cannot increase the root water content of plants under drought conditions, whereas spraying the sericin protein working solution can significantly increase the root water content of tomato plants under drought stress.

As shown in FIG. 2D, the relative decrease in plant biomass of the root fresh weight in the HS group (sprayed with distilled water) is slightly lower than that in the NS group (no spray of any substance), but the difference is not significant. In contrast, the relative decrease in plant biomass of the root fresh weight in the DS group (sprayed with sericin protein working solution) is significantly lower than that in both the HS group and the NS group. Therefore, the sericin protein working solution prepared in the disclosure significantly improves the drought tolerance of tomato plants and mitigated the impact of the drought environment on root biomass.

As shown in FIG. 2E, the relative decrease in plant biomass of the root dry weight in the HS group (sprayed with distilled water) is significantly lower than that in the NS group (no spray of any substance). The relative decrease in plant biomass of the root dry weight in the DS group (sprayed with sericin protein working solution) is significantly lower than that in the NS group (no spray of any substance) and also lower than that in the HS group (sprayed with distilled water). This indicates that, compared to the test group sprayed with no substance and the group sprayed with distilled water, spraying the sericin protein working solution significantly alleviates the impact of drought stress on both fresh weight and dry weight of root biomass, which demonstrates that spraying the sericin protein working solution can significantly reduce the impact of drought on root growth.

### (2) Effect of Sericin Protein Working Solution on Shoot Biomass and Shoot Water Content of Tomato Plants Under Drought Stress

The results are shown in FIG. 2F. The shoot fresh weight of tomato plants in the DS group (sprayed with sericin protein working solution) is significantly higher than that of tomato plants in both the NS group (no spray of any substance) and the HS group (sprayed with distilled water). As shown in FIG. 2G, the shoot dry weight of tomato plants in the DS group is also significantly higher than that of tomato plants in both the NS group and the HS group.

As shown in FIG. 2H, the shoot water content of tomato plants in the DS group is significantly higher than that of tomato plants in both the NS group and the HS group.

As shown in FIG. 2I, the relative decrease in plant biomass of the shoot fresh weight of tomato plants in the DS group is significantly lower than that of tomato plants in both the NS group and the HS group.

As shown in FIG. 2J, the relative decrease in plant biomass of the shoot dry weight of tomato plants in the DS group is slightly lower than that of tomato plants in both the NS group and the HS group.

The above results indicate that spraying the sericin protein working solution may alleviate the impact of drought stress on shoot biomass by increasing the shoot water content of the plants.

In summary, foliar spraying of the sericin protein working solution can significantly enhance the drought tolerance of tomato plants, mitigate the impact of drought on plants, and improve plant survival rate and biomass under drought stress. This demonstrates that the sericin protein working solution can be used as a spray agent to enhance plant drought tolerance, which lays a foundation for crop cultivation in arid environments.

Although some of the embodiments of the disclosure have been described, those skilled in the art, once aware of the basic inventive concept, may make additional modifications and changes to these embodiments.

Apparently, those skilled in the art may make various modifications and variations to the disclosure without departing from a spirit and a scope of the disclosure. Thus, if these modifications and variations of the disclosure fall within the scope of the equivalent technology of the disclosure, the disclosure is also intended to encompass these modifications and variations.

What is claimed is:

1. An application of a sericin protein working solution in tomato plant cultivation, wherein the sericin protein working solution is configured to improve a drought tolerance of a tomato plant;
wherein a preparation method of the sericin protein working solution comprises: fragmenting silkworm cocoons to obtain fragmented silkworm cocoons, boiling the fragmented silkworm cocoons in distilled water for 10 minutes (min) to 15 min to obtain a sericin protein stock solution;
diluting the sericin protein stock solution with distilled water to a concentration of 0.1 gram per liter (g/L) to 0.5 g/L, to obtain the sericin protein working solution; and an amount ratio of the silkworm cocoons to the distilled water is 5 grams (g) to 10 g: 200 milliliters (mL);
wherein the application comprises:
spraying the sericin protein working solution onto adaxial surfaces of leaves of the tomato plant when the tomato plant grows to a five-true-leaves and one-heart stage, with a spray volume of 10 mL/plant to 15 mL/plant;
wherein a spraying timing is set when the tomato plant grows to the five-true-leaves and one-heart stage, the spraying is performed at a time point when a cultivation environment changes from darkness to light; and
wherein the cultivation environment is repeated cycles of 16 hours (h) of light and 8 h of darkness.

2. The application of the sericin protein working solution in tomato plant cultivation as claimed in claim 1, wherein a concentration of the sericin protein working solution is 0.1 g/L.

3. The application of the sericin protein working solution in tomato plant cultivation as claimed in claim 1, wherein the sericin protein working solution is configured to improve a survival rate of the tomato plant under drought stress.

4. The application of the sericin protein working solution in tomato plant cultivation as claimed in claim 1, wherein the sericin protein working solution is configured to improve a root biomass and a root water content of the tomato plant under drought stress.

5. The application of the sericin protein working solution in tomato plant cultivation as claimed in claim 1, wherein the sericin protein working solution is configured to improve a shoot biomass and a shoot water content of the tomato plant under drought stress.

6. The application of the sericin protein working solution in tomato plant cultivation as claimed in claim 1, wherein the tomato plant is wild-type tomato Ailsa craig.
